SEQUENCE LISTING (2 sequences)

SEQ ID NO: 1 - protein, 687 aa, Homo sapiens
YFSGWFIKSSNEATNITPKHNMKAFLDELKAENIKKFLYNFTQIPHLAGTEQNFQLAKQIQSQWKEFGLDSAELAHYDVLLSYPNKTHPNYISIINEDGNEIFNTSLFEPPPPGYENVSDIVPSFSAFSPQGMPEGDLVHVNYARTEDFFKLERDMKINCSGKIVIARYRKVFRGNKVKNAQLAGAKGVILYSDPADYFAPGVKSYPDGWNLPGGGVQRGNILNLNGAGDPLTPGYPANEYAYRHGIAEAVGLPSIPVHPVGYYDAQKLLEKMGGSAPPDSSWRGSLKVSYNVGPGFTGNFSTQDRYVILGGHRDSWVFGGIDPQSGAAVVHETVRSFGTLKKEGWRPRRTILFASWDAEEFGLLGSTEWAEDNSRLLQERGVAYINADSSIEGEYTLRVDCTPLMYSLVYNLTKEVLKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYNISMKHPQEMKTYSLSFDSLFSAVKNFTEIASKFSERLQDFDKSNPILLRMMNDQLMFLERAFIDPLGLPDRPFYRHVIYAPSSHNKYAGESFPGIYDALFDIESKVDPSKAWGDVKRQISVAAFTVQAAAETLSEVA

SEQ ID NO: 2 - DNA, 2061 bp, Homo sapiens
tatttttcagggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgctgagctagctcattatgatgtcctgttgtcctacccaaataagacacatcccaactacatctcaataattaatgaagatggaaatgagattttcaacacatcattatttgaaccacctcctccaggatatgaaaatgtttcggatattgtaccatctttcagtgctttctctcctcaaggaatgccagagggtgatctagtgcatgttaactatgcacgaactgaagacttctttaaattggaacgggacatgaaaatcaattgctctgggaaaattgtaattgccagatataggaaagttttcagaggaaataaggttaaaaatgcccagctggcaggggccaaaggagtcattctctactcagaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatacgcttataggcatggaattgcagaggctgttggtcttccaagtattcctgttcatccagttggatactatgatgcacagaagctcctagagaaaatgggtggctcagcaccaccagatagcagctggagaggaagtctcaaagtgtcctacaatgttggacctggctttactggaaacttttctacacaggacagatatgtcattctgggaggtcaccgggactcatgggtgtttggtggtattgaccctcagagtggagcagctgttgttcatgaaactgtgaggagctttggaacactgaaaaaggaagggtggagacctagaagaacaattttgtttgcaagctgggatgcagaagaatttggtcttcttggttctactgagtgggcagaggataattcaagactccttcaagagcgtggcgtggcttatattaatgctgactcatctatagaaggtgaatatactctgagagttgattgtacaccactgatgtacagcttggtatacaacctaacaaaagaggtactgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacaatatttctatgaaacatccacaggaaatgaagacatacagtttatcatttgattcacttttttctgcagtaaaaaattttacagaaattgcttccaagttcagcgagagactccaggactttgacaaaagcaacccaatattgttaagaatgatgaatgatcaactcatgtttctggaaagagcatttattgatccattagggttaccagacagacctttttataggcatgtcatctatgctccaagcagccacaacaagtatgcaggggagtcattcccaggaatttatgatgctctgtttgatattgaaagcaaagtggacccttccaaggcctggggagatgtgaagagacagatttctgttgcagccttcacagtgcaggcagctgcagagactttgagtgaagtagcc

(19) United States
(12) Patent Application Publication
(10) Pub. No.: US 2004/0053283 A1
(43) Pub. Date: Mar. 18, 2004

(54) Gene 4

(76) Inventors: Colin Andrew Mclean Semple, Edinburgh (GB); Donald Robert Dunbar, Newhouse Lanarkshire (GB)

Correspondence Address: William M Blackstone, Intervet Inc Patent Department, 405 State Street, PO Box 318, Millsboro, DE, 19966, US

(21) Appl. No.: 10/399,157

(22) PCT Filed: Sep. 21, 2001
(86) PCT No.: PCT/EP01/10998

(30) Foreign Application Priority Data
Sep. 28, 2000 (EP) 00308551.1

(51) Int. Cl. C12Q 1/68; C07H 21/04; C12N 9/00; C12N 15/52

(52) U.S. Cl. 435/6; 435/69.1; 435/320.1; 435/325; 435/183; 530/350; 536/23.2

(57) ABSTRACT

A nucleotide acid sequence is provided encoding a protein having NAALAdase like activity NALADase activity has been found to be decreased in schizophrenic brain tissue. Its encoded protein can be used to screen for glutamate peptidase modulators.

The present invention relates to a newly identified DNA sequence which is localized in the proximity of a breakpoint on chromosome 11 involved in a balanced t(1;11)(q42.1;q14.3) translocation. The invention also relates to its encoded protein as well as to transformed cell lines.

Family, twin and adoption studies have convincingly demonstrated a significant genetic contribution to schizophrenia (McGuffin P et a 1995, Lancet 346:678-682, and references therein) and have driven studies directed at identification of this genetic component. Schizophrenia is a complex disease and the multifactorial and probable genetic heterogeneity of the condition complicates the application and interpretation of conventional linkage and association studies.

Previously, a balanced t(b 1;11)(q42.1;q14.3) translocation was reported that is associated with schizophrenia and other related mental illness in a large Scottish family (St Clair D et al 1990, Lancet 336:13-16). Mapping of the translocation breakpoint on chromosome 11, and the accompanying search for neighbouring genes has already been reported (Devon R S et al 1997, Am. J. Med. Genet. 74:82-90, 1998, Pyschiatr. Genet. 8:175-181).

Studies of the chromosomal breakpoint region have found two novel genes (DISC1 and DISC2; Millar J K et al 2000, Hum Mol Genet 9:1415-1423) that are directly disrupted by the translocation.

Psychiatric phenotypes linked with the translocation might be the result of position effects upon neighbouring genes on chromosome 11. It is therefor of importance to characterize genes on the chromosome 11 breakpoint region.

There have been various reports of the effects of translocation breakpoints exerting long range position effects on known disease genes up to 900 Kb away (Kleinjan D J and van Heyningen V 1998, Hum Mol Genet 7:1611-1611). It is therefore conceivable that one or more of the genes located around the breakpoint region may suffer alterations in their regulation as a result of the translocation. Alternatively it is possible that a disease associated allele of one of these genes is co-segregating with the translocation in the pedigree examined. Even over distances of several hundred kilobases significant linkage disequilibrium may be detected (Collins A et al 1999, Proc Natl Acad Sci USA, 96:15173-15177) and may be enhanced by the presence of a neighbouring translocation acting to reduce recombination.

We now have identified a novel gene which is thought to be involved in schizophrenia via its proximity to the breakpoint. The gene is found to be located at 704 kilobase pairs from the translocation breakpoint at chromosome 11 and it shows extensive homology with folate hydrolase (PMSA, prostate-specific membrane antigen). Functionally, PMSA has NAALADase activity and it is to be expected that Gene 4 also has such activity. NAALADase is an enzyme with glutamate carboxypeptidase activity.

Glutamate is the major excitatory neurotransmitter in mammalian brain and is required for normal brain function, acting through a number of receptors. It has been hypothesised that hypofunction of glutamatergic neurones has a pathological role in schizophrenia (Hirsch S R et al 1997, Pharmacol Biochem Behav 56:797-802), this hypothesis being strengthened by the fact that the NMDA receptor antagonist phencyclidine hydrochloride (PCP) can induce both positive and negative effects of schizophrenia. Reduced glutamate has been shown in schizophrenic cerebrospinal fluid and postmortem brain.

The enzyme hydrolyses the neuropeptide N-acetyl-L-aspartyl-L-glutamate (NAAG) to generate another neuropeptide, N-acetyl aspartate (NAA) and glutamate (see FIG. 1). NAALADase activity has been shown to be decreased in schizophrenic brain (prefrontal and hippocampal regions), as have the products NAA and glutamate (Tsai G et al 1995, Arch Gen Psychiatry 52:829-836 ). There are specific inhibitors of NAALADase, for example 2-(phosphonomethyl)pentanedioic acid (2-PMPA or GPI5000) that have been proposed for treatment of conditions caused by excessive glutamate (i.e. neuroprotective effect): stroke, ischemic brain injury, neuropatbic pain, spinal cord injury, amyotrophic lateral sclerosis (Slusher B S et al 1999, Nat Med 5:1396-1402; Whelan J 2000, Drug Discov Today 5:171-172). These inhibitors decrease the amount of glutamate and NAA presynaptically, but also increase the level of the substrate NAAG. As NAAG binds the inhibitory metabotropic glutamate receptor mGluR3, increased NAAG levels lead to a further inhibitory effect (Wroblewska B et al 1997, J Neurochem 69:174-181). 2-PNPA has been tested in a tissue culture model of cerebral ischemia where 10 μM had 85% protection from cellular injury. This protective effect is significant even one hour after the ischemia. 2-PNPA was also tested in vivo in a rat stroke model (middle ear cerebral artery occlusion). The inhibitor was well tolerated at levels that produced a high degree of neuronal protection.

The use of NAALADase agonists has not been discussed in the literature. By increasing the activity of NAALADase (using compounds that act on gene 4 protein) glutamate and NAA levels would be increased, and NAAG would be decreased (thus decreasing its inhibitory effect through mGluR3). In addition, NAALADase has also been shown to be a weak partial agonist of NMDA receptors (Pangalos MN et al 1999, J. Biol. Chem 274:8470-8483).

It will be clear that there is a great need for the elucidation of genes related to schizophrenia in order to unravel the various roles these genes may play in the disease process. A better knowledge of the genes involved in schizophrenia and the mechanism of action of their encoded proteins might help to create a better insight into the etiology of this psychiatric disorder and its underlying molecular mechanisms. This could eventually lead to improved therapy, selection of activity modulators and better diagnostic procedures.

The present invention provides such a gene which is located on chromosome 11. More specific, the present invention provides for a gene, called gene 4 whose cDNA sequence is partially shown in SEQ ID NO: 2.

The sequences of the present invention can be used to prepare probes or as a source to prepare synthetic oligonucleotides to be used as primers in DNA amplification reactions allowing the isolation and identification of the complete gene. The complete genetic sequence can be used in the preparation of vector molecules for expression of the protein in suitable host cells.

Using the sequence information provided herein, complete genes or variants thereof can be derived from cDNA or genomic DNA from natural sources or synthesized using known methods.

Thus, an additional embodiment of the invention is a method to isolate a gene comprising the steps of: a) hybridizing a DNA according to the present invention under stringent conditions against nucleic acids being RNA, (genomic) DNA or cDNA isolated preferably from tissues which highly express the DNA of interest; and b) isolating said nucleic acids by methods known to a skilled person in the art. The tissues preferably are from human origin. Preferably ribonucleic acids are isolated from brain. The hybridization conditions are preferably highly stringent.

According to the present invention the term “stringent” means washing conditions of 1×SSC, 0.1% SDS at a temperature of 65° C.; highly stringent conditions refer to a reduction in SSC towards 0.3×SSC, preferably 0.1×SSC.

As an alternative the method to isolate the gene might comprise gene amplification methodology using primers derived from the nucleic acid according to the invention. Complete cDNAs might also be obtained by combining clones obtained by e.g. hybridization with e.g. RACE cDNA clones.

Thus, the invention also includes the entire coding sequence part of which is indicated in SEQ ID NO: 2. Furthermore, to accommodate codon variability, the invention also includes sequences coding for the same amino acid sequence as the amino acid sequence disclosed herein and presented in SEQ ID NO: 1. Also portions of the coding sequence coding for individual domains of the expressed protein are part of the invention as well as allelic and species variations thereof. Sometimes, a gene is expressed in a certain tissue as a splicing variant, resulting in an altered 5′ or 3′ mRNA or the inclusion of an additional exon sequence. Alternatively, the messenger might have an exon less as compared to its counterpart. These sequences as well as the proteins encoded by these sequences all are expected to perform the same or similar functions and also form part of the invention.

The sequence information as provided herein should not be so narrowly construed as to require inclusion of erroneously identified bases. The specific sequence disclosed herein can be readily used to isolate the complete genes which in turn can easily be subjected to further sequence analyses thereby identifying sequencing errors.

Thus, in one aspect, the present invention provides for isolated polynucleotides encoding a glutamate carboxypeptidase, more specifically an N-acetyl-L-aspartyl-L-glutamate protease.

The DNA according to the invention may be obtained from cDNA. Alternatively, the coding sequence might be genomic DNA, or prepared using DNA synthesis techniques. The polynucleotide may also be in the form of RNA. If the polynucleotide is DNA, it may be in single stranded or double stranded form. The single strand might be the coding strand or the non-coding (anti-sense) strand.

The present invention further relates to polynucleotides which have at least 98% and even more preferably at least 99% identity with SEQ ID NO: 2. Such polynucleotides encode polypeptides which retain the same biological function or activity as the natural, mature protein. Alternatively, also fragments of the above mentioned polynucleotides which code for domains of the protein which still are capable of binding to substrates are embodied in the invention.

The percentage of identity between two sequences can be determined with programs such as DNAMAN (Lynnon Biosoft, version 3.2). Using this program two sequences can be aligned using the optimal alignment algorithm of Smith and Waterman (1981, J. Mol. Biol, 147:195-197). After alignment of the two sequences the percentage identity can be calculated by dividing the number of identical nucleotides between the two sequences by the length of the aligned sequences minus the length of all gaps.

The DNA according to the invention will be very useful for in vivo or in vitro expression of the novel protease according to the invention in sufficient quantities and in substantially pure form.

In another aspect of the invention, there is provided for a protein comprising the amino acid sequence encoded by the above described DNA molecules.

Preferably, the protein according to the invention comprises an amino acid sequence shown in SEQ ID NO: 1.

Also functional equivalents, that is polypeptides homologous to SEQ ID NO: 1 or parts thereof having variations of the sequence while still maintaining functional characteristics, are included in the invention.

The variations that can occur in a sequence may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities have been described. Amino acid replacements between related amino acids or replacements which have occurred frequently in evolution are, inter alia Ser/Ala, Ser/Gly, Asp/Gly, Asp/Asn, Ile/Val (see Dayhof, M. D., Atlas of protein sequence and structure, Nat. Biomed. Res. Found., Wash. D.C., 1978, vol. 5, suppl. 3). Based on this information Lipman and Pearson developed a method for rapid and sensitive protein comparison (1985, Science 227, 1435-1441) and determining the functional similarity between homologous polypeptides.

The polypeptides according to the present invention include the polypeptides comprising variants of SEQ ID NO: 1, i.e. polypeptides with a similarity of 98%, preferably 99% as compared to SEQ ID NO: 1. Also portions of such polypeptides still capable of conferring biological effects are included. Especially portions which still bind to substrates form part of the invention. Such portions may be functional per se, e.g. in solubilized form or they might be linked to other polypeptides, either by known biotechnological ways or by chemical synthesis, to obtain chimeric proteins. Such proteins might be useful as therapeutic agent in that they may substitute the gene product in individuals with aberrant expression of the Gene 4 gene.

The sequence of the gene may also be used in the preparation of vector molecules for the expression of the encoded protein in suitable host cells. A wide variety of host cell and cloning vehicle combinations may be usefully employed in cloning the nucleic acid sequence coding for the Gene 4 protein of the invention or parts thereof. For example, useful cloning vehicles may include chromosomal, non-chromosomal and synthetic DNA sequences such as various known bacterial plasmids and wider host range plasmids and vectors derived from combinations of plasmids and phage or virus DNA.

Vehicles for use in expression of the genes or a part thereof comprising a peptidase activity containing domain of the present invention will further comprise control sequences operably linked to the nucleic acid sequence coding for the protein. Such control sequences generally comprise a promoter sequence and sequences which regulate and/or enhance expression levels. Of course control and other sequences can vary depending on the host cell selected.

Suitable expression vectors are for example bacterial or yeast plasmids, wide host range plasmids and vectors derived from combinations of plasmid and phage or virus DNA. Vectors derived from chromosomal DNA are also included. Furthermore an origin of replication and/or a dominant selection marker can be present in the vector according to the invention. The vectors according to the invention are suitable for transforming a host cell.

Recombinant expression vectors comprising the DNA of the invention as well as cells transformed with said DNA or said expression vector also form part of the present invention.

Suitable host cells according to the invention are bacterial host cells, yeast and other fungi, plant or animal host such as Chinese Hamster Ovary cells or monkey cells. Thus, a host cell which comprises the DNA or expression vector according to the invention is also within the scope of the invention. The engineered host cells can be cultured in conventional nutrient media which can be modified e.g. for appropriate selection, amplification or induction of transcription. The culture conditions such as temperature, pH, nutrients etc. are well known to those ordinary skilled in the art.

The techniques for the preparation of the DNA or the vector according to the invention as well as the transformation or transfection of a host cell with said DNA or vector are standard and well known in the art, see for instance Sambrook et al., Molecular Cloning: A laboratory Manual. 2^{nd} Ed., Cold Spring Harbor Laboratory, Cold Spring Harbor, N.Y., 1989.

The proteins according to the invention can be recovered and purified from recombinant cell cultures by common biochemical purification methods including ammonium sulfate precipitation, extraction, chromatography such as hydrophobic interaction chromatography, cation or anion exchange chromatography or affinity chromatography and high performance liquid chromatography. If necessary, also protein refolding steps can be included.

Gene 4 gene products according to the present invention can be used for the in vivo or in vitro identification of novel substrates or analogs thereof. For this purpose e.g. protease assay studies can be performed with cells transformed with DNA according to the invention or an expression vector comprising DNA according to the invention, said cells expressing the gene 4 gene products according to the invention. Alternatively also the gene 4 gene product itself or the substrate-binding domains thereof can be used in an assay for the identification of functional substrates or analogs for the gene 4 gene products.

Methods to determine glutamate protease activity of expressed gene products in vitro and in vivo assays to determine biological activity of gene products are well known (see e.g. Robinson M B et al 1987 J Biol Chem 262:14498-14506). In general, the amount of ³H labeled glutamate released from ³H labeled NAAG under hydrolyzing conditions (e.g. in 50 mM Tris-HCl or 5 mM HEPES (HBSS solution) buffer in 15 min at 37° C.) can be measured. Substrates and products can be resolved by ion-exchange liquid chromatography.

The following example is illustrative for the invention and should in no way be interpreted as limiting the scope of the invention.

### LEGENDS TO THE FIGURES

FIG. 1 Hydrolysis of NAAG to NAA and glutamate

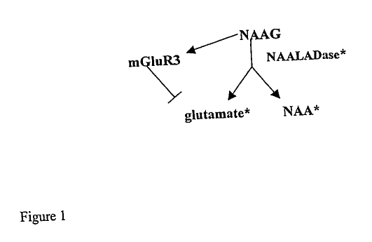

The asterisks (on NAALADase, NAA and glutamate) represent a demonstrated reduction of levels in schizophrenic brain. NAAG selectively activates the metabotropic glutamate receptor mGluR3 which in turn, decreases glutamate release.

### EXAMPLES

### Example 1

A genomic BAC clone contiguous (contig) was made by searching previously mapped chromosome 11 genomic clone sequences against the publicly available High Throughput Genomic (HTG) Sequence section of the EMBL database by BLAST (Altschul et al 1997, Nucl Acids Res 25:3389-3402). Four BAC sequences were used intially based on their mapping to chromosome 11q14: AP000827, AP000648, AP0000684 and AP000651 to identify further BACS to extend the clone contig. When the genomic clone contig was searched against all available public sequence databases, several similarities to transcribed sequences were found. On BACS AP000827, AP000648, a match to NAALADase II was found. Another gene, on BAC AC024234, was found, which is very closely related to Prostate Specific Membrane Antigen (PSMA, accession number NM_{—}004476). A nucleotide acid sequence of part of the gene is shown in SEQ ID NO: 2. The sequence codes for an amino acid sequence as identified in SEQ ID NO: 1.

1. A polynucleotide comprising encoding the amino acid SEQ ID NO: 1.

2. The polynucleotide according to claim 1 said polynucleotide comprising the nucleic acid sequence SEQ ID NO: 2.

3. The polynucleotide according to claim 1 or 2 said polynucleotide consisting of a nucleic acid sequence encoding the amino acid sequence SEQ ID NO: 1.

4. The polynucleotide according to claims 1-3 said polynucleotide consisting of the nucleic acid sequence SEQ ID NO: 2.

5. A recombinant expression vector comprising the DNA according to claims 1-4.

6. A polypeptide encoded by the polynucleotide according to claims 1-4 or the expression vector according to claim 5.

7. A cell transfected with a polynucleotide according to claims 1-4 or the expression vector according to claim 5.

8. The cell according to claim 7 which is a stable transfected cell which expresses the polypeptide according to claim 6.

9. Use of the polynucleotide according to claims 1-4 or an expression vector according to claim 5, a cell according to claims 7 or 8 or a polypeptide according to claim 6 in a screening assay for identification of new drugs.
